(19) United States
(12) Patent Application Publication
(10) Pub. No.: US 2016/0101140 A1
(43) Pub. Date: Apr. 14, 2016

(54) Method for Processing Rosaceous Kernels

(71) Applicant: Richard Hwang, New Taipei City (TW)

(72) Inventor: Richard Hwang, New Taipei City (TW)

(21) Appl. No.: 14/514,645

(22) Filed: Oct. 15, 2014

(30) Foreign Application Priority Data
Oct. 9, 2014 (TW) 103135238

(51) Int. Cl. A61K 36/736; A23L 2/52; A23L 1/36; A23L 1/302; A61K 36/889; A61K 31/7028

(52) U.S. Cl. CPC A61K 36/736; A61K 36/889; A61K 31/7028; A23L 1/362; A23L 1/302; A23L 2/52; A23V 2002/00

(57) ABSTRACT

A method for processing rosaceous kernels includes the steps of preparing a predetermined amount of rosaceous kernels, heating the rosaceous kernels to a predetermined temperature range for a predetermined time, lowering a temperature of the rosaceous kernels to a normal temperature, and making the rosaceous kernels edible for users to intake Vit B17 contained in the rosaceous kernels. Through the heating process, the beta-glucosidase contained in the rosaceous kernels can be destroyed completely, but the Vit B17 still stays in the rosaceous kernels.

This application claims the benefit of Taiwanese Patent Application Serial No. 103135238, filed Oct. 9, 2014, the subject matter of which is incorporated herein by reference.

### BACKGROUND OF INVENTION

1. Field of the Invention

The invention relates to a method for processing rosaceous kernels, and more particularly to a heating method that can destroy completely the beta-glucosidase inside the rosaceous kernels without losing any of the Vitamin B17 (Vit B17) within the rosaceous kernels.

2. Description of the Prior Art

Thousands of artificial harmful chemical substances resulted from industrial pollutions would be definitely dangerous to human bodies. If the source for damaging the body is never stopped, non-stop body healing would highly possibly lead to a growth of a tumor if the immune system and the pancreatic enzymes can't provide enough defense weapons to reduce the damage as well as the healing. Generally, the tumor would grow at the damaged portion of the body. At this time, if there is enough Vit B17 in the foods, a second defense line can then be formed.

If the function of the immune system is low and insufficient Vit B17 is taken, then the cancer would grow gradually and silently. By comparing the rate of occurrence of cancers between the developed countries and the under-development countries, it is found that the rate for the developed countries leaded by the United States is much higher than that for the under-development countries. By having the usage of mobile phones as a flag, the cancers caused by the mobile phone's usage are highly related to the handling of the mobile phone. In another instance, the carcinogens in the foods (for example, Japanese yellow pickled radish) would also cause the stomach, the colorectal and the like digestive tract to be damaged. In addition, the electromagnetic waves for most of the electronic apparatuses would hurt specific portion or whole of the body. Further, extra sunshine may also damage the skin. All the aforesaid locations that are harmed by various causes would be obviously vulnerable to a cancer's growth.

It is Dr. Harold Manner, chairman of the biology department of Loyola University, to prove that the Vit B17 requires pancreatic enzymes to help destroy the surface layer of the tumor cell, such that the Vit B17 can attack the tumor cell directly. Contemporarily, a specific requested experiment, supervised by Dr. Kanematsu Sugiura, was performed in the Sloan Kettering Memorial Hospital of Rockefeller group, one of the most remarkable cancer-research hospitals in New York. After five years of devotion, Sugiura concluded the following findings.

1. Vit B17 had anti-cancer activity.

2. Vit B17 inhibited secondary tumors in mice.

3. Vit B17 can lessen the pain caused by the cancer.

4. Vit B17 can inhibit an occurrence of a cancer.

5. Vit B17 can promote the health.

In the same Sugiura's report, it was mentioned that the thorough recovery from the tumors in the experiment was never seen in any prior test of other chemotherapeutic drugs. The same Sugiura's experiments were performed again by Dr. Elizabeth Stockert and Dr. Llyod Schoen, both of which were appointed by the hospital authority. These two researchers confirmed the findings of Sugiura. Schoen also confirmed Manner's conclusion. All these efforts contribute to establish a therapy standard for applying the Vit B17 and kemelrition enzymes.

In 1950's, a biochemist Ernst T. Krebs, Jr. extracted a new Vit B17, named also laetrile or amygdalin, from apricot kernels. The Vit B17, synthesized into crystals to be used by human body, is a chemical compound formed by a molecule of hydrogen cyanide, a molecule of benzaldehyde with an analgesic effect, and two molecules of glucose. Though the aforesaid two components are individually severe toxic, yet the Vit B17 binding these two toxic components was shown to be stable and non-toxic. Krebs also found that only a glucose enzyme of beta-glucosidase can break the chemical bond between the aforesaid two toxic components. Though the same enzyme can be found in human body, yet a pretty high concentration of this enzyme is found in the area around the tumor. Hence, as the beta-glucosidase enzymes secreted by the tumor cells break the chemical bonds of Vit B17, the toxic hydrogen cyanide and phenylacetaldehyde would be released to provide enhanced toxicity for killing the tumor cells.

In addition, the Vit B17 is a water-soluble and non-toxic carbohydrate compound existing widely in at least 800 plants in the natural world, in which most of the plants are edible. Among these plants containing plenty Vit B17, seeds of non-citrus fruits, such as cherries, nectarines, peaches, plums, apples, apricots and so on are usually seen in the marketplace. In particular, the seed of apricot can have a content of 2-4 wt %. Generally, normal rosaceous kernels, the kernels of bitter almonds for example, are usually bitter-tasted and toxic, in which the source of the bitter taste in the kernel is the Vit B17.

References for self-defense mechanism in the kernels of the rosaceae plants (the bitter almond for example) include at least the following.

A. Hydrogen cyanide, website:

http://www.plbio.life.ku.dk/Formidling/Tjek_paa_biotek/˜/media/Plbio/formidling/Gymnasielaererkursus/LIFE_vejledning_Lotus %20HCN.ashx

B. Hydrogen cyanide, website:

http://www.ch.ic.ac.uk/rzepa/mim/environmental/html/hcn.htm

C. Wikipedia—Amygdalin, website:

http://en.wikipedia.org/wiki/Amygdalin

In the art, the debittering technology for the kernels of bitter almonds can have the following two methods.

1. A water-dipping or water-boiling method for debittering: As kernels of the bitter almonds are debittered, the beta-glucosidase of the kernels would dissolve into the water or be destroyed by high temperatures, such that the chemical reaction for breaking the cyanide bongs is inhibited and thus the toxic hydrogen cyanide can't be produced. Anyhow, the Vit B17 contained in the kernels of the bitter almonds would be discharged into the water a little bit.

2. A water or solvent dipping method: The organic solvents used for this method would then contain the beta-glucosidase and the Vit B17 shall be recycled. Also, the kernels of the bitter almonds free of the organic solvent are now no more bitter and toxic as well. Namely, the Vit B17 original in the kernels for the bitter taste is gone with the organic solvent, and the accompanying toxicity does no more exist.

### SUMMARY OF THE INVENTION

Accordingly, it is the primary object of the present invention to provide a method for processing rosaceous kernels that requires no extra additive and doesn't apply the heating by direct dipping. Also, by applying the method of the present invention for processing the rosaceous kernels, the beta-glucosidase would be ridded off, but the Vit B17 can stay.

In the present invention, the method for processing rosaceous kernels includes the steps of processing a predetermined amount of rosaceous kernels, heating the rosaceous kernels to a predetermined temperature range for a predetermined time, lowering a temperature of the rosaceous kernels to a normal temperature, and making the rosaceous kernels edible for users to intake Vit B17 contained in the rosaceous kernels. Through the heating process, the beta-glucosidase contained in the rosaceous kernels can be destroyed completely, but the Vit B17 still stays in the rosaceous kernels.

It is another object of the present invention to provide a method for processing rosaceous kernels, in which wet-heating or steam-boiling is applied to heat the rosaceous kernels so as to get rid of the toxic beta-glucosidase but stay the Vit B17 in the rosaceous kernels.

All these objects are achieved by the method for processing rosaceous kernels described below.

### BRIEF DESCRIPTION OF THE DRAWINGS

The present invention will now be specified with reference to its preferred embodiment illustrated in the drawings, in which:

FIG. 1 is a flowchart of a first embodiment of the method for processing rosaceous kernels in accordance with the present invention;

FIG. 2 is a flowchart of a second embodiment of the method for processing rosaceous kernels in accordance with the present invention;

FIG. 3 is a flowchart of a third embodiment of the method for processing rosaceous kernels in accordance with the present invention;

FIG. 4 is a flowchart of a fourth embodiment of the method for processing rosaceous kernels in accordance with the present invention; and

FIG. 5 is a flowchart of a fifth embodiment of the method for processing rosaceous kernels in accordance with the present invention.

### DESCRIPTION OF THE PREFERRED EMBODIMENT

The invention disclosed herein is directed to a method for processing rosaceous kernels. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

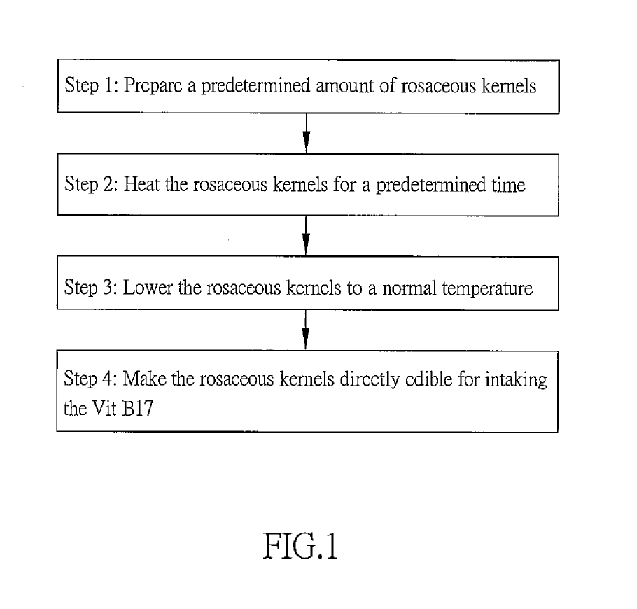

Referring now to FIG. 1, a flowchart of a first embodiment of the method for processing rosaceous kernels in accordance with the present invention is shown. This embodiment is mainly targeted to directly heat the rosaceous kernels without adding any water, solvent or additive, such that the bitter taste and the toxic beta-glucosidase of the rosaceous kernels can be completely ridded off, but the Vit B17 thereof can stay.

In the present invention, the method for processing rosaceous kernels includes the following steps 1 to 4.

Step 1: Prepare a predetermined amount of rosaceous kernels.

Step 2: Heat the rosaceous kernels to a predetermined temperature range for a predetermined time.

Step 3: Lower a temperature of the rosaceous kernels to a normal temperature.

Step 4: Make the rosaceous kernels edible for users to intake the Vit B17 contained in the rosaceous kernels. In this first embodiment, the rosaceous kernels are made to be directly served or edible after a further compensation work.

The merchandise name of the Vit B17 is the bitter almond glycosides, a chemical compound of the glycosides group. For the Vit B17 is a chemical compound consisted of two sugar molecules, a benzaldehyde molecule and a cyanide molecule and can be hydrolyzed inside the human body to produce a hydrogen cyanide and a benzaldehyde, from which the toxicity follows. Catalyzed by specific enzymes, the bitter almond glycosides (Vit B17) can undergo two decomposition reactions. One is the maltase to perform partly decomposition for generating a D-glucose and a mandelonitrile glucoside (C6H5CH(CN)O.C6H11O5), an isomer of a benzeneacetonitrile glucoside. Another is the bitter almond enzyme to perform decomposition for generating a benzaldehyde, a cyanide such as a hydrogen cyanide (HCN), and two molecule of glucoses. Namely, such a decomposition reaction can also be performed in the bitter almond so as to induce the benzaldehyde and the cyanide inside the bitter almond. It is the reason why, while the bitter almond is chewed, the Vit B17 is decomposed by the glucose glycosides enzyme so as to produce the toxic hydrogen cyanide.

Further, it is to say why, while the bitter almond glycosides (Vit B17) react with the tumor cells of human body, a cyanide component is released. As the beta-glucosidase enzymes secreted by the tumor cells break the chemical bonds of Vit B17, the hydrogen cyanide and the benzaldehyde would be released to kill the tumor cells. However, if health cells are involved, a rhodanese as a protective enzyme would be released. It is interesting that the rhodanese exists in all-over the health cells, but not in the tumor cells. Therefore, if the Vit B17 meets the health cells, the rhodanese secreted by the health cells would break the Vit B17 at proper points so that no toxic hydrogen cyanide can be produced. It is found that by-products of the aforesaid reaction include the thiocyanate and the benzoic acid, both of which can nutrition the health cells with extra thereof to be expelled out of the body through the urine. Accordingly, toxicity of the hydrogen cyanide can be decomposed so as not to have the cyanide to hurt the health cells. Also, for the tumor cells won't secrete the rhodanese, and thus the cyanide would kill the tumor cells.

In the first embodiment of the present invention as shown in FIG. 1, the rosaceous kernels are embodied as the kernels of the bitter almonds. In order to maintain the Vit B17 in the kernels of the bitter almonds, the aforesaid Step 1 to Step 4 of the processing method shall be performed to provide direct heating, in a manner of non-dipping in a water, a solvent or any additive, for destroying completely the beta-glucosidase contained in the kernels of the bitter almonds. Further, for the melt point of the Vit B17 is about 223° C.˜226° C., so the beta-glucosidase has already been destroyed (by heated up to about 48° C.˜56° C. for a predetermined time) prior to reaching the melt point of the Vit B17, but the Vit B17 in the kernels of the bitter almonds can be stayed. Upon such an arrangement, a nontoxic kernel of the bitter almond with the Vit B17 can be provided for powdering, water-mixing, grinding, or mixing with other foods.

In the Step 2 of the method of the present invention, the predetermined temperature range is preferably ranged from 70° C. to 120° C., more preferably ranged from 70° C. to 80° C. for such a temperature range would get rid of the toxicity without ruining the content of the Vit B17 by the boiling water. In the present invention, the preset time is at least five minute. The rosaceous kernels of the present invention can be selected from the group of bitter almond kernels, peach seed kernels, apple seeds, date seed kernels, plum seed kernels, cherry seed kernels and nectarine seed kernels.

Another application is firstly to perform the aforesaid Step 1 and the Step 2, and then to perform a Vit B17-extraction process whether the temperature is reduced or not. In natural rosaceous kernels, the Vit B17 and the beta-glucosidase won't “meet” each other anyway. It seems that, in the rosaceous kernel, the Vit B17 and the beta-glucosidase are stayed in separate rooms. However, in the art, the conventional method for extracting the Vit B17 generally includes the steps of grinding, dissolving, water-dipping and stirring. It is quite possible that during these conventional steps these two components would meet and then induce a hydrolyzing reaction to consume a substantial amount of the Vit B17. Though the water-dipping would dissolve the hydrolyzed hydrogen cyanide HCN into water or solvent, the extracted Vit B17 can still contain no hydrogen cyanide. However, it is true, during the extracting process, that the loss or pre-hydrolyzing of Vit B17 is inevitable. After the Step 2, the activity of the beta-glucosidase is removed, and thus further stirring, grinding or water-dipping won't lead to hydrolyze anymore, such that the loss of the Vit B17 into water can be reduced. Namely, thereby, the extract performance of the Vit B17 can be enhanced.

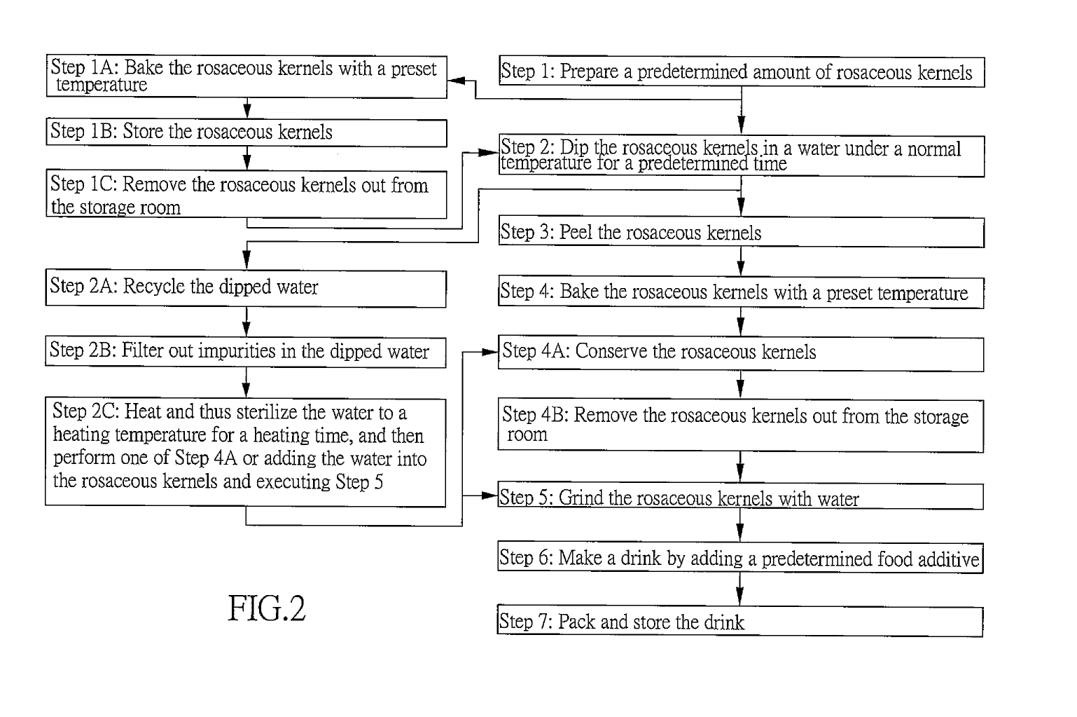

Referring now to FIG. 2, a flowchart of a second embodiment of the method for processing rosaceous kernels in accordance with the present invention is shown. In this embodiment, the method of the present invention is to prepare the rosaceous kernels as a drink and further includes the following steps 1 to 7.

Step 1: Prepare a predetermined amount of rosaceous kernels. In this embodiment, the Step 1 further includes the following Steps 1A to 1C.

Step 1A: Bake fresh rosaceous kernels with another preset temperature that won't over-heat the rosaceous kernels.

Step 1B: Store the rosaceous kernels into a storage room.

Step 1C: Remove the rosaceous kernels out from the storage room.

Step 2: Dip the rosaceous kernels in a water under a normal temperature for a predetermined time. In this embodiment, the Step 2 further includes the following Steps 2A to 2C.

Step 2A: Recycle the water that dips the rosaceous kernels.

Step 2B: Filter the water so as to remove impurities in the water.

Step 2C: Heat and thus sterilize the water to a heating temperature for a heating time and then perform one of the Step 3, the following Step 4A, and a step of adding the water into the rosaceous kernels and executing the following Step 5.

Step 3: Peel the rosaceous kernels.

Step 4: Bake the rosaceous kernels with a preset temperature. In this embodiment, the Step 4 further includes the following Steps 4A to 4B.

Step 4A: Conserve the rosaceous kernels in the storage room.

Step 4B: Remove the rosaceous kernels out from the storage room.

Step 5: Grind the rosaceous kernels with another water.

Step 6: Make a drink of the rosaceous kernels by adding a predetermined food additive.

Step 7: Package the drink and store it into the storage room.

Namely, in the second embodiment of the present invention, the rosaceous kernels of the Step 1 can belong to the bitter almonds (wet) or the sweet almonds. In the Step 1, either the Step 1A or the Step 2, in which the Step 1A is to directly heat the rosaceous kernels to the predetermined temperature range (preferably 70° C.˜120° C.). Namely, the beta-glucosidase in the fresh rosaceous kernels would be destroyed completely by the heating, but the Vit B17 therein can be stayed. Further, perform the Step 1B and the Step 1C to bake and then store the bitter almonds or the sweet almonds, or further perform the Step 2 to dip the rosaceous kernels of the Step 1 in the water under the normal temperature for days long so as to have the Vit B17 of the bitter almonds or the sweet almonds to dissolve into the water. Then, perform the Step 2A or the Step 3, in which, while in performing the Step 3, the rosaceous kernels are peeled to enhance the color and the taste of the drink. Thereafter, perform the Step 4 to bake again by either a low temperature or a high temperature. Them, perform the Step 4A and the Step 4B to store the baked rosaceous kernels into the storage room for future usage.

Between the Step 2 and the Step 3, i.e. after the dipping of the rosaceous kernels but prior to the peeling, apply the Step 2A and the Step 2B to recycle the dipping water by filtering out the impurities therein. Further, the Step 2C is applied to heat the recycled water to a temperature between 75° C. and 120° C. for above 4˜6 minutes to process the sterilizing. Also, thereby the beta-glucosidase dissolved in the water can be destroyed so as to fail the activity of the accompanying enzymes. The recycled water can undergo the Step 4A and Step 4B to be stored into the storage room for future usage, or to be directly applied as the water of the Step 5. In this usage, the filtered water having the Vit B17 can be used a part of the drink of the rosaceous kernels. In performing the Step 6, the drink of the rosaceous kernels can be added by relevant food additives, such as the sugar. In performing the Step 7, the drink is packed and then stored into the storage room. In the second embodiment of the method for processing rosaceous kernels as shown in FIG. 2, the Step 1A, the Step 1B and the Step 1C can be directly skipped. Also, the Step 2A, the Step 2B and the Step 2C can be directly skipped as well.

Namely, by compared to the conventional method for making the drink of the rosaceous kernels, either the bitter almonds or the sweet almonds, the Step 2 thereof is to discharge the dipped water through sewage treatment equipments. Hence, the related cost for this conventional process is high, and also the Vit B17 dissolved in the water can't be recycled and thus wasted forever. On the other hand, in the present invention, the dipped water having the Vit B17 is filtered and then fed back as the water for grinding the rosaceous kernels, such that the content of the Vit B17 in the drink of the rosaceous kernels can be increased, and thus the nutrition and the taste of the drink can be superior to that of the conventional drink of the rosaceous kernels.

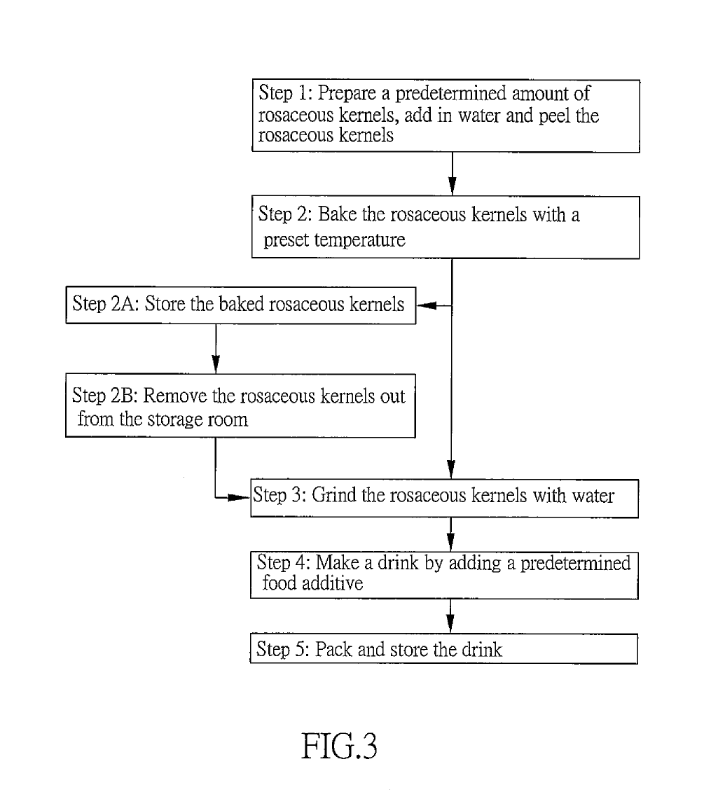

Referring now to FIG. 3, a flowchart of a third embodiment of the method for processing rosaceous kernels in accordance with the present invention is shown. In this embodiment, the method for processing rosaceous kernels is to form a drink of the rosaceous kernels and includes the following steps 1 to 5.

Step 1: Prepare a predetermined amount of rosaceous kernels, add a water to the rosaceous kernels, and peel the rosaceous kernels.

Step 2: Bake the rosaceous kernels with a preset temperature. In this embodiment, the Step 2 further includes the following Steps 2A to 2B.

Step 2A: Store the baked rosaceous kernels into a storage room.

Step 2B: Remove the rosaceous kernels out from the storage room and then perform the following Step 3.

Step 3: Grind the rosaceous kernels with another water.

Step 4: Make a drink of the rosaceous kernels by adding a predetermined food additive.

Step 5: Package the drink and store it into the storage room.

In this embodiment, after the baking of the Step 2 no matter if the temperature is cooled, the activity of the beta-glucosidase in the kernels has already been destroyed completely. Then, the rosaceous kernels are now ready for the material for extracting the Vit B17. The third embodiment of the method for processing rosaceous kernels in accordance with the present invention is characterized on that, the baking of the Step 2 can destroy completely the activity of the beta-glucosidase in the rosaceous kernels, but the Vit B17 therein can be stayed. Thereby, possible hydrolyzing of the Vit B17 by the resided beta-glucosidase can be avoided in extracting the Vit B17. Therefore, a further loss of the Vit B17 therefrom can be inhibited.

Namely, in the aforesaid Step 2A and Step 2B, prior to any drying, conserving or reworking, the baking temperature of the rosaceous kernels is raised to a temperature preferably between 70° C. and 120° C., so that the toxicity of the rosaceous kernels can be removed, but the function of the Vit B17 can be kept.

As shown in FIG. 3, in the third embodiment of the present invention, the rosaceae for the kernel can be a bitter almond (wet) or a sweet almond. The drink of the bitter almond or the sweet almond can be made by performing the aforesaid Step 1 through the Step 5. In order to stay the Vit B17 of the rosaceous kernels, the rosaceous kernels are firstly peeled in the Step 1 by adding the water, and, the Step 2, a predetermined baking temperature is applied to destroy the beta-glucosidase in the kernels. Then, in the Step 2A and Step 2B, the baked rosaceous kernels is moved to storage, and alternatively the baked rosaceous kernels directly undergo the Step 3 to be ground into a water mixture. In the Step 4, the mixture is added by appropriate food additives for enhancing the taste such as the sugar so as to form the drink of the rosaceous kernels. Then, the drink is moved to be conserved in the storage room in the Step 5. In this third embodiment, the Step 2A and the Step 2B can be skipped.

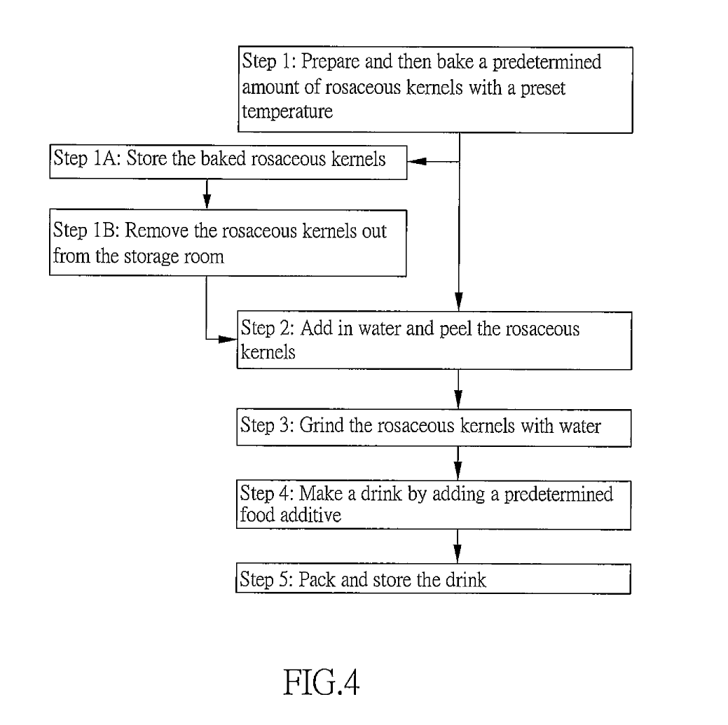

Referring now to FIG. 4, a flowchart of a fourth embodiment of the method for processing rosaceous kernels in accordance with the present invention is shown. In this embodiment, the method for processing rosaceous kernels is to form a drink of the rosaceous kernels and includes the following steps 1 to 5.

Step 1: Prepare and then bake a predetermined amount of rosaceous kernels with a preset temperature, in which the preset temperature is preferably ranged from 70° C. to 120° C. In this embodiment, the Step 1 further includes the following Steps 1A to 1B.

Step 1A: Store the baked rosaceous kernels into a storage room.

Step 1B: Remove the rosaceous kernels out from the storage room and then perform the following Step 2.

Step 2: Add a water to, or add a water to and peel, the rosaceous kernels.

Step 3: Grind the rosaceous kernels with another water.

Step 4: Make a drink of the rosaceous kernels by adding a predetermined food additive.

Step 5: Package the drink and store it into the storage room.

As shown in FIG. 4, in the fourth embodiment of the present invention, the rosaceae for the kernel can be a bitter almond (wet) or a sweet almond. The drink of the bitter almond or the sweet almond can be made by performing the aforesaid Step 1 through the Step 5. In order to stay the Vit B17 of the rosaceous kernels, the rosaceous kernels are firstly heated to a preset temperature range so as to destroy the beta-glucosidase (Step 1). Then, in the Step 1A and the Step 1B, the baked rosaceous kernels are stored for further usage, or alternatively the baked rosaceous kernels are directly added by a water and then peeled in the Step 2. Further, in the Step 3, the peeled rosaceous kernels are ground into a liquid mixture with another water. In the Step 4, food additives for taste adjustment such as the sugar can be added into the liquid mixture so as to form a drink of the rosaceous kernels. Finally, in the Step 5, the drink is further packaged and stored into the storage space. In this fourth embodiment, the Step 1A and the Step 1B can be skipped.

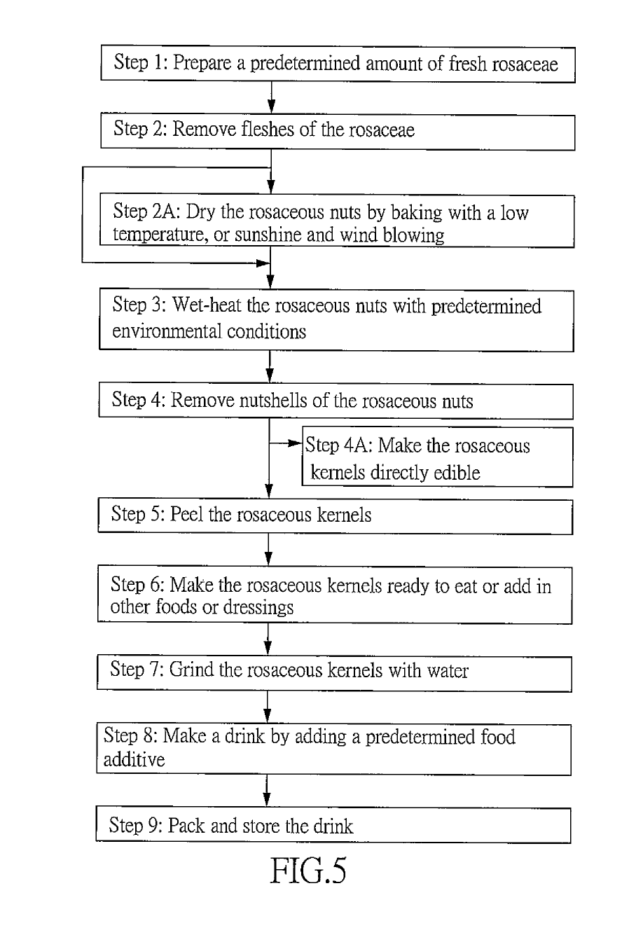

Referring now to FIG. 5, a flowchart of a fifth embodiment of the method for processing rosaceous kernels in accordance with the present invention is shown. In this embodiment, the method for processing rosaceous kernels is to form a drink of the rosaceous kernels and includes the following steps 1 to 9.

Step 1: Prepare a predetermined amount of fresh rosaceae.

Step 2: Remove fleshes of the rosaceae so as to obtain rosaceous nuts. In this embodiment, the Step 2 further includes the following Step 2A.

Step 2A: Dry the rosaceous nuts by one of baking with a low temperature, sun shining and wind blowing.

Step 3: Wet-heat the rosaceous nuts with predetermined environmental conditions.

Step 4: Remove nutshells of the rosaceous nuts so as to obtain rosaceous kernels. In this embodiment, the Step 4 further includes the following Step 4A.

Step 4A: Make the rosaceous kernels directly edible.

Step 5: Peel the rosaceous kernels.

Step 6: Make a solid food ready to eat of the rosaceous kernels or another solid food by adding a predetermined food additive.

Step 7: Grind the rosaceous kernels with another water.

Step 8: Make a drink of the rosaceous kernels by adding a predetermined food additive.

Step 9: Package the drink and store it into the storage room.

As shown in FIG. 5, in the fifth embodiment of the present invention, the rosaceae can be a bitter almond or a sweet almond. The process for making a drink of the bitter almond or the sweet almond shall include the aforesaid Step 1 through the Step 9. Firstly, in the Step 1, a predetermined amount of the fresh rosaceae is prepared. Then, in the Step 2, the fleshes of the rosaceae are removed so as to obtain the rosaceous nut (i.e. the kernel with the nutshell). In the Step 2A, the defleshed rosaceae, i.e. the nut, is baked by a low temperature, or dried by sunshine or wind. In order to retain the Vit B17 in the rosaceous kernel, in the Step 3, the rosaceous nut is wet-heated with predetermined environmental conditions so as to destroy the beta-glucosidase therein. Further, in the Step 4, the nutshell of the dried rosaceous nut is removed to expose the kernel thereinside, and, in the Step 4A, the rosaceous kernel can be ready to eat, or alternative the Step 5 is performed to peel the rosaceous kernel. Further, in the Step 6, the kernel is now ready to eat or can be adjusted by adding in other foods or dressings. In the Step 7, the pure rosaceous kernel is ground with a water to form a liquid mixture. In the Step 8, a predetermined food additive is added to the liquid mixture so as to obtain a good-taste and good-smell drink. Finally, in the Step 9, the drink is packaged and then stored into the storage room.

In the present invention, the predetermined environmental conditions for the wet-heating of the Step 3 can be selected from the group of (1) a temperature of about 121° C., a pressure of about 1.05 kg/cm² and a time duration of 20 minutes; (2) a steam of 100° C. and a time duration of 20 minutes; and, (3) a steam of 100° C. and a time duration of 60 minutes.

In this fifth embodiment, the predetermined food additive of the Step 8 can be a sugar. Besides, in this fifth embodiment, the Step 2A and the Step 4A can be individually skipped.

While the present invention has been particularly shown and described with reference to a preferred embodiment, it will be understood by those skilled in the art that various changes in form and detail may be without departing from the spirit and scope of the present invention.

What is claimed is:

1. A method for processing rosaceous kernels, comprising the steps of:
(Step 1) preparing a predetermined amount of rosaceous kernels;
(Step 2) heating the rosaceous kernels to a predetermined temperature range for a predetermined time in a non-dipping manner;
(Step 3) lowering a temperature of the rosaceous kernels to a normal temperature; and
(Step 4) making the rosaceous kernels edible for users to intake Vit B17 contained in the rosaceous kernels;
wherein, in the Step 2, beta-glucosidase contained in the rosaceous kernels is completely destroyed, but the Vit B17 therein is stayed.

2. The method for processing rosaceous kernels of claim 1, wherein, in the Step 2, the predetermined temperature range is a range of 70° C.˜120° C.

3. The method for processing rosaceous kernels of claim 1, wherein, in the Step 2, the predetermined time is at least 5 minutes.

4. The method for processing rosaceous kernels of claim 1, wherein the rosaceous kernels are selected from the group of bitter almond kernels, peach seed kernels, apple seeds, date seed kernels, plum seed kernels, cherry seed kernels and nectarine seed kernels.

5. A method for processing rosaceous kernels, comprising the steps of:
(Step 1) preparing a predetermined amount of rosaceous kernels;
(Step 2) dipping the rosaceous kernels in a water under a normal temperature for a predetermined time;
(Step 3) peeling the rosaceous kernels;
(Step 4) baking the rosaceous kernels with a preset temperature;
(Step 5) grinding the rosaceous kernels with another water; and
(Step 6) making a drink of the rosaceous kernels by adding a predetermined food additive.

6. The method for processing rosaceous kernels of claim 5, wherein the Step 1 further includes the steps of:
(Step 1A) baking fresh rosaceous kernels with another preset temperature that won't over-heat the rosaceous kernels;
(Step 1B) storing the rosaceous kernels into a storage room; and
(Step 1C) removing the rosaceous kernels out from the storage room and performing the Step 2; and
wherein the Step 4 further includes the steps of, after the baking:
(Step 4A) conserving the rosaceous kernels in the storage room; and
(Step 4B) removing the rosaceous kernels out from the storage room and performing the Step 5.

7. The method for processing rosaceous kernels of claim 6, wherein the Step 2 further includes the steps of:
(Step 2A) recycling the water that dips the rosaceous kernels;
(Step 2B) filtering the water so as to remove impurities in the water; and
(Step 2C) heating and thus sterilizing the water to a heating temperature for a heating time, and then performing one of the Step 3, the Step 4A, and a step of adding the water into the rosaceous kernels and executing the Step 5.

8. The method for processing rosaceous kernels of claim 7, wherein, in the Step 2C, the heating temperature is ranged from 75° C. to 85° C., and the heating time is ranged from 4 to 6 minutes.

9. A method for processing rosaceous kernels, comprising the steps of:
(Step 1) preparing a predetermined amount of rosaceous kernels, adding a water to the rosaceous kernels, and peeling the rosaceous kernels;
(Step 2) baking the rosaceous kernels with a preset temperature;
(Step 3) grinding the rosaceous kernels with another water; and
(Step 4) making a drink of the rosaceous kernels by adding a predetermined food additive;
wherein, after the Step 2, the rosaceous kernels are formed as a material for extracting Vit B17;
wherein, in the Step 2, activity of beta-glucosidase contained in the rosaceous kernels is completely destroyed, but the Vit B17 therein is stayed, such that the loss of the Vit B17 caused by the beta-glucosidase possibly resided in the rosaceous kernels hydrolyzing the Vit B17 during a following extracting process of the Vit B17 can be avoided.

10. The method for processing rosaceous kernels of claim 9, wherein the Step 2 further includes the steps of:
(Step 2A) storing the baked rosaceous kernels into a storage room; and
(Step 2B) removing the rosaceous kernels out from the storage room and performing the Step 3;
wherein, prior to any process of drying, conserving and reworking, the rosaceous kernels are baked with the present temperature of the Step 2 so as to stay the Vit B17 contained in the rosaceous kernels;
wherein with the present temperature of the Step 2 is ranged from 70° C. to 120° C.

11. A method for processing rosaceous kernels, comprising the steps of:
(Step 1) preparing and then baking a predetermined amount of rosaceous kernels with a preset temperature;
(Step 2) adding a water to, or adding a water to and peeling, the rosaceous kernels;
(Step 3) grinding the rosaceous kernels with another water; and
(Step 4) making a drink of the rosaceous kernels by adding a predetermined food additive.

12. The method for processing rosaceous kernels of claim 11, wherein the Step 1 further includes the steps of:
(Step 1A) storing the baked rosaceous kernels into a storage room; and
(Step 1B) removing the rosaceous kernels out from the storage room and performing the Step 2;
wherein the preset temperature of the Step 1 is ranged from 70° C. to 120° C.

13. A method for processing rosaceous kernels, comprising the steps of:
(Step 1) preparing a predetermined amount of fresh rosaceae;
(Step 2) removing fleshes of the rosaceae so as to obtain rosaceous nuts;
(Step 3) wet-heating the rosaceous nuts with predetermined environmental conditions; and
(Step 4) removing nutshells of the rosaceous nuts so as to obtain rosaceous kernels.

14. The method for processing rosaceous kernels of claim 13, wherein the Step 2 further includes a step 2A of drying the rosaceous nuts by one of baking with a low temperature, sun shining and wind blowing.

15. The method for processing rosaceous kernels of claim 13, wherein the predetermined environmental conditions for the wet-heating of the Step 3 are selected from the group of:
(1) a temperature of about 121° C., a pressure of about 1.05 kg/cm² and a time duration of 20 minutes;
(2) a steam of 100° C. and a time duration of 20 minutes; and
(3) a steam of 100° C. and a time duration of 60 minutes.

16. The method for processing rosaceous kernels of claim 13, wherein the Step 4 further includes a Step 4A of making the rosaceous kernels directly edible or edible after adding other foods or additives.

17. The method for processing rosaceous kernels of claim 13, further including, posterior to the Step 4, the steps of:
(Step 5) peeling the rosaceous kernels;
(Step 6) grinding the rosaceous kernels with another water; and
(Step 7) making a drink of the rosaceous kernels by adding a predetermined food additive.
